(19) United States
(12) Patent Application Publication
(10) Pub. No.: US 2009/0156422 A1
(43) Pub. Date: Jun. 18, 2009

(54) DEVICE AND METHOD TO DETECT ANALYTES

(75) Inventors: Chamindie Punyadeera, Eindhoven (NL); Ron Van Lieshout, Mierlo (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/302,910

(22) PCT Filed: May 24, 2007
(86) PCT No.: PCT/IB2007/051959

§ 371 Date: Dec. 1, 2008

(30) Foreign Application Priority Data
Jun. 2, 2006 (EP) 06114901.9

(51) Int. Cl. C40B 30/04; G01N 33/00; C12M 1/34; G01N 33/53

(52) U.S. Cl. 506/9; 422/68.1; 435/287.1; 436/501; 435/7.1

(57) ABSTRACT

The present invention relates to a device for and method of detecting analytes in samples, using optimized concentrations of Fc receptor-antibody complexes being immobilized on solid supports.

The invention relates to a detection device for detecting an analyte in a sample, which relies on complexes of an analyte sensor receptor molecule and an analyte sensor molecule being attached to a support.

The invention further relates to a method of detecting an analyte in a sample using said detection device.

Moreover, the invention relates to an apparatus comprising the detection device in accordance with the invention.

Miniature detection devices have been used in various chemical and biochemical diagnostic and synthetic applications. Such detection devices are used e.g. for lateral flow tests and drug of abuse tests with a particular focus on point-of-care-diagnostics.

US patent application 2003/0198967 A1 describes array-based detection devices in which ProteinA is coupled via an acyl fluoride functionality to a solid substrate. In a second step, an antibody is bound to the ProteinA-loaded support. This arrangement is then used for detecting analytes in a sample which specifically bind to the ProteinA-bound antibody. In this device setup, ProteinA is used as an analyte sensor receptor molecule while the antibody constitutes the analyte sensor molecule.

However, immobilization of analyte sensor molecules in a functional configuration on a support, improvement of signal to noise ratio during analyte detection and reduction of the amounts of the commonly costly analyte sensor receptor molecules and analyte sensor molecules remain critical factors for the development of further miniaturised effective detection devices.

It is an object of the present invention to provide a detection device and methods for determining the presence of an analyte in a sample to be tested.

It is a further object of the present invention to provide a detection device which allows efficient detection of an analyte in a sample while improving the amounts of analyte sensor receptor molecule and/or analyte sensor molecule to be used.

In order to achieve the above-defined objects, a detection device as defined in independent claim 1 and a method of detection as defined in independent claim 14 are provided.

According to an exemplary embodiment of the invention, a detection device is provided which comprises at least one support, at least one analyte sensor receptor molecule which is positioned on and/or within at least a part of said at least one support, and at least one analyte sensor molecule which is associated with said at least one analyte sensor receptor molecule, with a molar ratio of said at least one analyte sensor molecule and said at least one analyte sensor receptor molecule being between approximately 2:1 and approximately 1:10,000.

In another exemplary embodiment of the present invention, the molar ratio of said at least one analyte sensor molecule and said at least one analyte sensor receptor molecule is between approximately 1.9:1 and approximately 1:1000, between approximately 1.8:1 and approximately 1:750, between approximately 1.7:1 and approximately 1:500, between approximately 1.6:1 and approximately 1:250, between approximately 1.5:1 and approximately 1:150, between approximately 1.4:1 and approximately 1:125, between approximately 1.3:1 and approximately 1:100, between approximately 1.3:1 and approximately 1:90, between approximately 1.2:1 and approximately 1:80, between approximately 1.1:1 and approximately 1:70, between approximately 1:1 and approximately 1:60, between approximately 1:1.1 and approximately 1:50, between approximately 1:1.2 and approximately 1:40, between approximately 1:1.3 and approximately 1:30, preferably between approximately 1.4:1 and approximately 1:20, between approximately 1:1.5 and approximately 1:15 or between approximately 1:1.5 and approximately 1:10.

Yet another exemplary embodiment of the present invention relates to a detection device of the aforementioned characteristics, wherein the number of the analyte sensor molecules per μm² surface area of the at least one support is between approximately 50 and 250,000, between approximately 100 and approximately 100,000, between approximately 150 and approximately 75,000, between approximately 200 and approximately 50,000, between approximately 250 and approximately 25,000, between approximately 300 and approximately 21,000, between approximately 350 and approximately 18,000, between approximately 400 and approximately 15,000, between approximately 450 and approximately 12,000, between approximately 500 and approximately 11,000, between approximately 550 and approximately 9000 or between approximately 600 and approximately 8000.

In yet another embodiment of the present invention, the detection device comprises a number of analyte sensor receptor molecules per μm² surface area of the at least one support between approximately 500 and 250,000, between approximately 1000 and approximately 100,000, between approximately 1500 and approximately 50,000, between approximately 5000 and approximately 25,000, between approximately 6000 and approximately 20,000, between approximately 6500 and approximately 16,000, between approximately 7000 and approximately 15,000, between approximately 7500 and approximately 13,000 or between approximately 8000 and approximately 12,000.

In another embodiment, the number of analyte sensor receptor molecules and analyte sensor molecules per μm² surface area of support is selected to conform with above specified molar ratios.

In one embodiment of the present invention, the support of the detection device may be a solid substrate being selected from the group comprising porous or non-porous materials, including polymeric materials, glasses, ceramics, gels, ion material, non-wovens, metals, filters, membranes and composites thereof.

In a particularly preferred embodiment, membranes are used which are preferably made of nylon, nitrocellulose, PVDF, Polyethersulfone etc.

In one embodiment, the present invention relates to a detection device in which the analyte sensor receptor molecules are capable of binding analyte sensor molecules in a functional conformational arrangement. According to the invention, such a detection device comprises at least one analyte sensor molecule which is capable of specifically interacting with the analyte sensor receptor molecule by means of one binding site and with an analyte of interest by means of a second binding site, with binding to the analyte by the second binding site not being substantially influenced by the concomitant binding of the analyte sensor receptor molecule via the first binding site.

In one aspect of the present invention, the analyte sensor receptor molecules are capable of binding to the Fc portion of an antibody.

Thus, in one embodiment the analyte sensor receptor molecules may be selected from antibodies which specifically recognize the Fc portion of another antibody or preferably from a ProteinA of Staphylococcus aureus, ProteinG of streptococci of the C or G strains, or recombinant ProteinA/G. A particularly preferred embodiment uses recombinant ProteinA/G as the analyte sensor receptor molecule.

In one embodiment, the detection device uses analyte sensor molecules which are selected from aptamers, protein receptors, enzymes, antigens, ligands and haptens and particularly preferably antibodies.

One embodiment of the invention relates to a detection device in which the analyte sensor receptor molecule is disposed on a support, which may be a membrane such as a nitrocellulose membrane, by means of a covalent or non-covalent interaction. In the case of a covalent interaction, the analyte sensor receptor molecule may be linked to the support, which may be a membrane, by way of at least one chemical linkage. For this purpose, the support may provide at least one functional chemical group which is capable of establishing a chemical bond between the support and the analyte sensor receptor molecule. In a preferred embodiment, this functional group comprises a COOH group.

Yet another embodiment of the present invention relates to a detection device in which the analyte sensor molecule binds to the analyte sensor receptor molecules by way of covalent or non-covalent interaction. In the case of a non-covalent interaction, binding of the analyte sensor molecule to the analyte sensor receptor molecule may be mediated by the affinity between two molecules which results from the complementarity of the three dimensional structures of the analyte sensor molecule receptor and the analyte sensor molecule.

In yet another embodiment, the detection device can be treated with a solution which is capable of reducing, preventing and/or removing non-specific binding of components of the sample to be tested to the support, the analyte sensor receptor molecule and/or the analyte sensor molecule. In one embodiment of the present invention, a detection device has therefore been treated with a blocking solution which may comprise a compound selected from the group comprising BSA, HSA, FSA, Casein, Fc tails and/or detergents before contacting the sample to be tested.

The invention is also directed to a method of detecting at least one analyte in at least one sample, comprising the steps of:
providing at least one detection device as described above,
contacting said at least one detection device with at least one sample comprising at least one analyte,
optionally washing said at least one detection device with a solution capable of removing non-bound or non-specifically bound sample components,
detecting a specific interaction between said at least one analyte sensor molecule and at least one analyte of said at least one sample.

For detecting the interaction between an analyte of a sample and an analyte sensor molecule, the analyte may be modified with a detectable marker.

Such methods according to an object of the invention can be used, inter alia, in clinical analysis, identification of novel drugs, blood analysis, drug discovery, structure-functional research, forensics, testing of environmental samples, chemical exposure, testing of small molecule libraries, cell based assays, etc.

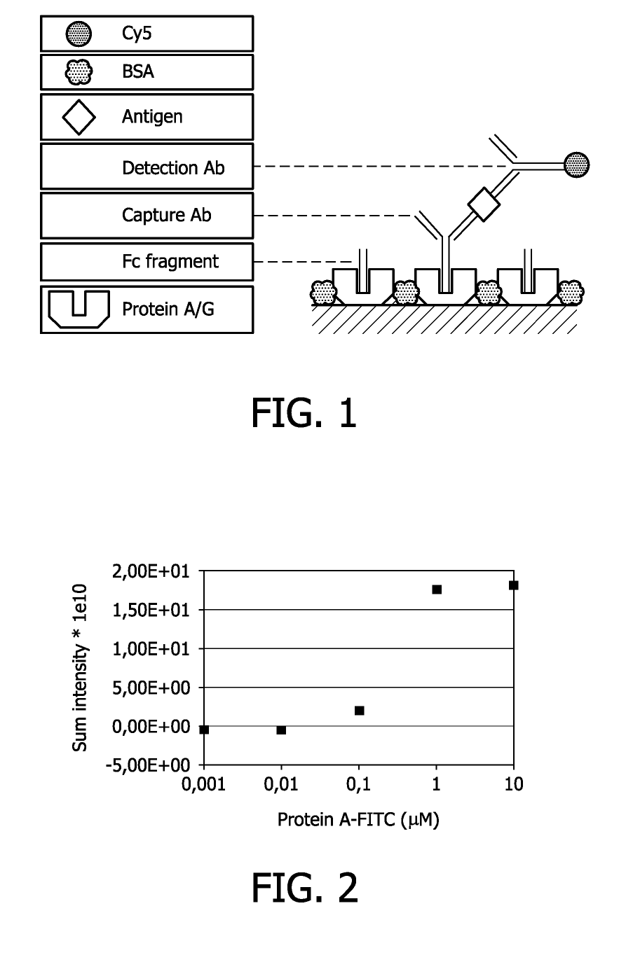

FIG. 1 is a schematic presentation of a detection device in accordance with the invention, which shows an embodiment in which ProteinA/G is bound to a solid membrane. The ProteinA/G coated membrane assembly is blocked with BSA and Fc fragments. Furthermore, the picture depicts binding of an antibody (analyte sensor molecule) to ProteinA/G and a bound analyte being coupled to a detectable Cy5 marker.

FIG. 2 shows the determination of the optimal concentration of a coating of ProteinA/G applied to a nitrocellulose membrane as described in Example 1.

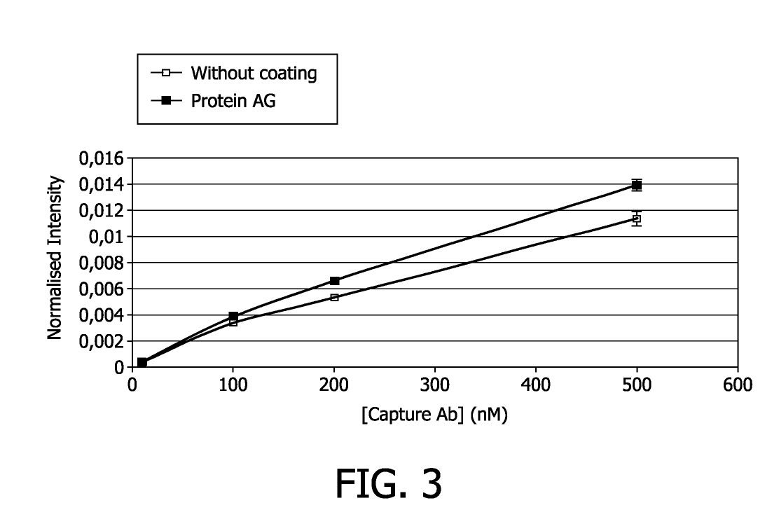

FIG. 3 shows the binding capacity of Cy5 labelled rabbit-anti-mouse IgG antibody bound to a ProteinA/G coated membrane versus a non-coated membrane.

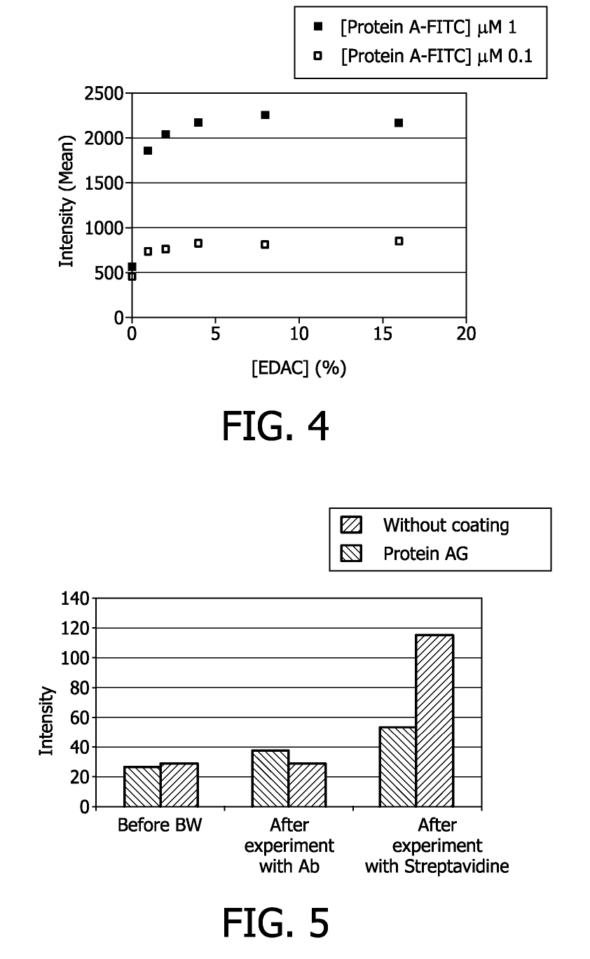

FIG. 4 shows the binding capacity of ProteinA/G as a function of EDAC.

FIG. 5 shows that a ProteinA/G coated membrane can be blocked with a blocking solution comprising Biotine labelled Rabbit-anti-Mouse antibody.

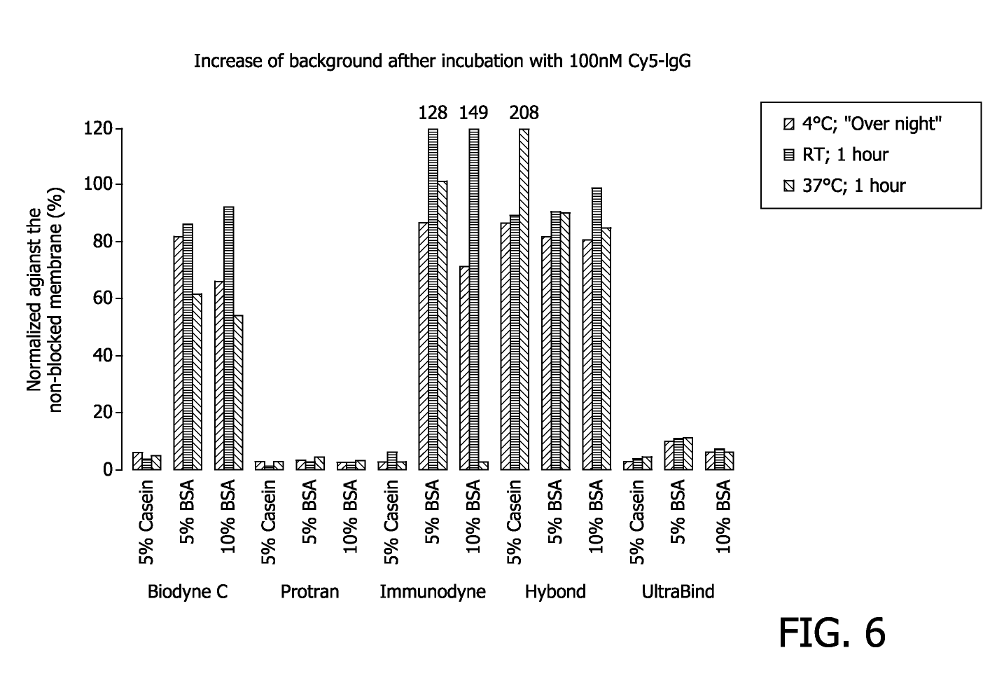

FIG. 6 shows the blocking efficiency of blocking solutions comprising BSA or casein for different membranes.

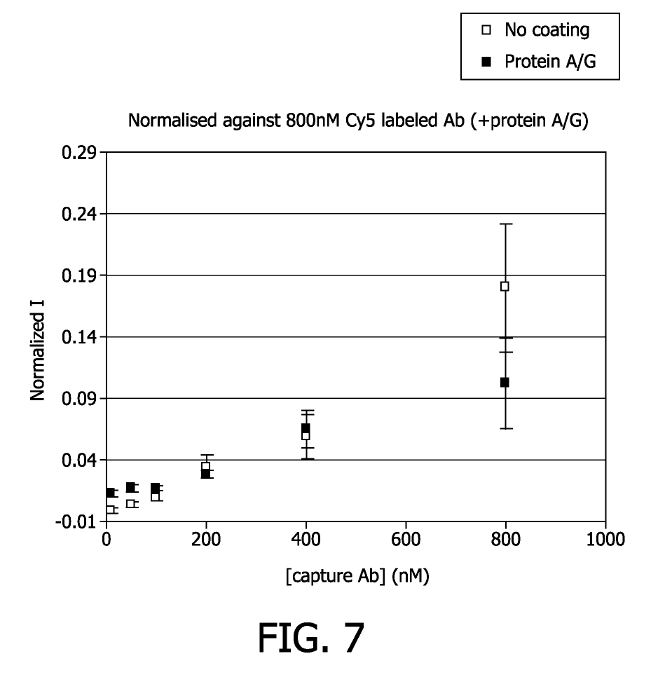

FIG. 7 shows the increased sensitivity of analyte detection at distinct molar ratios of captured antibody (analyte sensor molecule) to ProteinA/G (analyte sensor receptor molecule).

The present invention, in one embodiment, is directed to a detection device comprising:
at least one support,
at least one analyte sensor receptor molecule which is positioned on and/or within at least a part of said at least one support, and
at least one analyte sensor molecule which is associated with said at least one analyte sensor receptor molecule,
wherein the molar ratio of said at least one analyte sensor and said at least one analyte receptor molecule is between approximately 2:1 and approximately 1:10,000.

In the past, analyte sensor molecules such as, for example, antibodies have been attached to solid substrates such as, for example, micro-arrays, beads etc. However, if an analyte sensor molecule such as an antibody, which can be used to detect analytes in a sample, is directly attached to a solid support it may be that the analyte sensor molecule is bound to the support in a non-functional confirmation. This occurs if e.g. the binding site, which would be required for detecting the analyte in a sample, is not accessible because linkage to the solid support takes place via this binding site.

To resolve these difficulties, it has been considered to first dispose an analyte sensor receptor molecule on a support which then binds the analyte sensor molecule in a functional confirmation, which means that the analyte sensor receptor molecule interacts with one binding site within the analyte sensor molecule while a second binding site of the analyte sensor molecule which is required for binding to the analyte to be detected is substantially not affected by the interaction between the analyte sensor receptor molecule and the analyte sensor molecule. An example of such conformationally functional interactions between analyte sensor receptor molecules and analyte sensor molecules is the coating of a substrate with e.g. streptavidine while the analyte sensor molecule is coupled to biotine. The analyte sensor molecule then interacts via the biotine with the analyte sensor receptor molecule being disposed on the substrate which in this case is strepdavitine. This allows orienting the analyte sensor molecule in a functional confirmation on a support and thereby maintaining the binding sites of the analyte sensor molecule that are required for detection of the analyte in a sample that is freely accessible.

The inventors of the present invention have surprisingly found that a detection device comprising:
at least one support,
at least one analyte sensor receptor molecule which is positioned on and/or within at least a part of said at least one support, and
at least one analyte sensor molecule which is associated with said at least one analyte sensor receptor molecule,

wherein the molar ratio of said at least one analyte sensor and said at least one analyte receptor molecule is between approximately 2:1 and approximately 1:10,000 allows for an efficient and sensitive detection of analytes within a sample, while at the same time keeping the amount of analyte sensor molecules and analyte sensor receptor molecules comparatively low.

Thus, the present invention is directed in one aspect to detection devices comprising a support upon which at least one analyte sensor receptor molecule is disposed. This analyte sensor receptor molecule interacts with at least one analyte sensor molecule, keeping the latter in a functional conformation being capable of reacting with an analyte in a sample while being bound at the same time to the analyte sensor receptor molecule. As according to the present invention the analyte sensor molecules and the analyte sensor receptor molecules are present on the support in distinct ratios and/or numbers, an optimal balance between the number of molecules to be disposed and efficient analyte detection is achieved.

Supports useful in the present invention are, in embodiment, solid substrates. Substrates can be made from porous or non-porous materials being capable of allowing deposition of an analyte sensor molecule on at least part of the surface of the substrate. If the substrate is of e.g. porous character, the analyte sensor receptor molecules may not only be disposed on at least a part of the surface of the porous support, but also within at least a part of the porous solid support. Substrates which are useful for the purposes of the present invention are of course known from the state of the art, and a person skilled in the art will understand these substrates to be fabricated from among others polymeric materials, glasses, ceramics, gels, natural fibres, fibres, silicones, metals, non-wovens, filter materials, membranes and composites thereof.

A preferred embodiment uses membranes as a solid support. These membranes are preferably made from nylon, nitrocellulose or PVDF.

Such membranes can be commercially obtained under the trade names Protran, Ultrabind, Immunodyne, Hybond and Biodyne. The Biodyne membrane, which is a negatively charged nylon membrane (due to the presence of carboxyl groups) available from Pall, is particularly preferred.

Of course, solid supports can be fabricated in all shapes and sizes, depending on the particular use. Examples include plates, sheets, films, threads, spots etc. Preferred, but not required shapes are those with a flat planar surface such as a membrane, a filter or a microplate that can be preferably handled by an automated diagnostic system.

The dimension may vary but membranes having a thickness between 1 to 15 mm, between 2 to 12 mm, between 3 to 11 mm, between 4 to 10 mm, between 4 to 10 mm or around 8 mm and/or a pore size of 0.1 to 1 μm, between 0.2 and 0.8 μm, 0.3 and 0.7 μm and 0.4 to 0.6 μm are preferred in one embodiment. This is also valid if the membrane is e.g. one of the aforementioned negatively charged nylon membranes.

Analyte sensor receptor molecules useful in the present invention are capable of being disposed on a support as described above, while providing a binding site for an analyte sensor molecule keeping the latter in a functional conformation.

An analyte sensor receptor molecule thus can be a compound, complex, ligand or reagent that is capable of attaching or coupling a variety of biological or chemical materials to a support. The analyte sensor receptor molecule can be a protein, an enzyme, a carbohydrate, a nucleic acid, an oligonucleotite, a polynucleotite, an aptamer, a hapten, a drug, a dye, a small organic molecule, a cell, a cell fragment, a receptor or cell surface binding agents etc. Examples of analyte sensor receptor molecules include e.g. Streptavidine or Biotine, FLAG-Epitope, myc-Epitope, Al-tag, GST-tag, His-Tag, Maltose-binding protein tag etc.

One of the embodiments relates to a detection device which can be used in immuno-assays and thus relies on the use of antibodies as analyte sensor molecules, the analyte sensor receptor molecules being, in this embodiment, preferably so-called Fc receptors.

An Fc receptor is a molecule being capable of binding to the Fc portion of an antibody. In the precise context of an antibody as the analyte sensor molecule, binding of an Fc receptor to the Fc portion of the antibody ensures that the antibody (which can also be designated as capture antibody) is bound to the analyte sensor receptor molecule via the Fc portion, with the variable regions of the antibody which mediate specific binding to the analyte to be detected remaining freely accessible for this latter interaction.

Exemplary analyte receptor molecules in the case of Fc receptors comprise, in a preferred embodiment, proteins such as ProteinA of Staphylococcus aureus, ProteinG of Streptococci of strains C and G or recombinant ProteinA/G. Other exemplary Fc receptors can comprise antibodies themselves as long as these antibodies have been raised against the Fc portion of another antibody. Of course, Fc receptors can also e.g. comprise aptamers or other proteins as long as these molecules have been raised to specifically recognize the Fc portion of an antibody.

ProteinA/G, which is a product of a gene fusion of the SC binding domains of ProteinA and G, is particularly preferred. ProteinA/G has a broader binding specificity for antibodies than either ProteinA or ProteinG. It thus combines to all human IgG subclasses IgA, IgE, IgM and IgD. The binding preferences of ProteinA and G can e.g. be taken from the textbook Harlow and Lane, Antibodies: A laboratory manual, Cold Spring Harbor Laboratory Press, 1988 (e.g. page 617).

Analyte sensor molecules which are useful in the present invention are molecules that can be bound by the aforementioned analyte sensor receptor molecules in a functional confirmation, meaning that the analyte sensor molecule while being bound to the analyte sensor receptor molecule retains the potential for specifically interacting with the target structure which is commonly designated as the analyte.

If a sample comprising various analytes is incubated with an analyte sensor molecule, the analyte sensor molecule will preferentially interact with the analyte which it is specific for, and thus lead to a specific interaction that can subsequently be detected. Analyte sensor molecules which are useful for the purposes of the present invention, thus can be molecules that, while being bound to an analyte sensor receptor molecule as described above, are still capable of interacting specifically with another molecule. Thus, analyte sensor molecules can be proteins, enzymes, receptors, ligands, aptamers, DNA fragments, antigens, haptens, cells, cellular fragments, small molecules, nucleotide sequences and antibodies.

If, as described above, an Fc receptor is used as the analyte sensor receptor molecule, the analyte sensor molecule should provide an Fc tail. While this of course means that analyte sensor molecules may be antibodies which in this case may also be designated as capture antibodies, analyte sensor molecules are by no means restricted to this latter class of proteins. Any type of analyte sensor molecule that is capable of binding to or interacting specifically with another molecule and which has been fused to an Fc region can thus be used as an analyte sensor molecule if an Fc receptor is to be used as the analyte sensor receptor molecule. For example, a small molecule, an aptamer, a nucleotide sequence, a ligand etc. can be fused, e.g. via a chemical crosslink, to an Fc region and thus be brought into contact with one of the aforementioned Fc receptors.

In one embodiment of the invention, antibodies are preferred as the analyte sensor molecule, given that they provide an Fc portion that can interact with one of the aforementioned Fc receptors and another binding site which is specific for another molecule and which is clearly separated from the Fc portion of the antibody so that interaction of the antibody with the Fc receptor will have substantially no effect on the interaction between the antibody and the molecular structure it is specific for. If antibodies are used as analyte sensor molecules, they may also be designated as capture antibodies.

Antibodies to be used as analyte sensor molecules may be of any origin, including mouse, human, rat, chicken, sheep, goat etc. and comprise all types of antibodies which are commonly known in the art, such as monoclonal antibodies, polyclonal antibodies, chimeric antibodies, humanized antibodies etc.

An overview of different types of antibodies which can be used are found in Harlow and Lane (vide supra). Furthermore, all types of antibody subclasses such as the aforementioned IgA, IgE, IgM, IgG, IgD etc., may be used. Reference in this context is again made to Harlow and Lane (vide supra). Of course, the person skilled in the art will be aware that selection of a certain antibody may require using a certain Fc receptor as the analyte sensor receptor molecule.

In a particularly preferred embodiment of the present invention, an Fc receptor such as ProteinA, ProteinG or particularly ProteinA/G is used as the analyte sensor receptor molecule, antibodies are used as the analyte sensor molecule and a membrane which may be a nylon membrane having advantageously the above-mentioned dimensions as concerns thickness and pore size is used for a detection device in accordance with the invention.

The analyte sensor receptor molecule can be disposed on and/or within at least a part of any of the aforementioned supports via covalent or non-covalent linkage to the support.

A covalent linkage between the support and the analyte sensor receptor molecule means that a chemical bond is formed. For the purposes of a covalent linkage, this support can be functionalized in order to provide functional chemical groups that allow formation of a chemical linkage between the support and the analyte sensor receptor molecule.

If thus e.g. an Fc receptor is used as an analyte sensor receptor molecule and should be covalently coupled to the membrane, a membrane may be used that provides functional groups such as carboxyl groups, amide groups, hydroxyl groups, sulfhydryl groups etc.

These groups may then be cross-linked either directly to an Fc receptor or using a linker that may be homo- or hetero-bifunctional.

Thus, supports can be activated for providing a chemical linkage to the analyte receptor molecule by e.g. coating an inert solid substrate with a polymer having e.g. acyl fluoride functionalities. Other covalent attachment chemistries are also applicable but not limited to anhydrides, epoxides, aldehydes, hydrazides, acyl azides, aryl azides, diazo compounds, benzophenone, carbodiimide, imidoesters, isothiocyanates, NHS esters, CNBr, maleimides, tosylates, tresyl chloride, maleic acid anhydrides and carbonyldiimidazole.

In preferred embodiments, a carbodiimide functionality may be used for establishing a link between the support and the analyte sensor receptor molecule, such as an Fc receptor.

For this purpose, a negatively charged nylon membrane such as a Biodyne C membrane which comprises COOH groups can be pretreated with a linker such as EDAC (N-(3-Dimethylaminopropyl)-N′-ethylcarbodiimide hydrochloride). This active intermediate then reacts with the amino group of an analyte sensor receptor molecule such as an Fc receptor.

In a particular embodiment of the present invention in which, as mentioned above, an Fc receptor such as ProteinA/G and antibodies as well as a membrane are used, linkage of the Fc receptor such as ProteinA may take place by using a low concentration of EDAC.

Typically, these concentrations of EDAC will vary between 0 to 25% by weight of EDAC, with 0.5 to 10% by weight, 0.5 to 8% by weight, 0.5 to 4% by weight, 0.5 to 2% by weight and particularly 1% by weight being preferred.

If non-covalent interaction is used to dispose the analyte sensor receptor molecule on the support, mechanisms such as absorption, hydrophobic interaction etc., may be used.

The interaction between the analyte sensor receptor molecule and the analyte sensor molecule may also be established using covalent or non-covalent interaction.

A non-covalent interaction will typically rely on the binding affinity between the analyte sensor receptor molecule and the analyte sensor molecule, being a consequence of their complementary three-dimensional structures. Typical examples of these interactions are for example the interaction between streptavidine (analyte sensor receptor molecule) and biotin (part of the analyte sensor molecule), Fc receptor (analyte sensor receptor molecule) and Fc portion of the analyte sensor molecule.

Of course, interaction between the analyte sensor receptor molecule and the analyte sensor molecule may be established using covalant linkage. For these purposes, homo and/or hetero-bi and/or multifunctional cross linking agents may be used. Typical cross-linking agents include, but are not limited to, those already mentioned in Harlow et Lane (vide supra): including, but not limited to bis (sulfosuccinimid) bis(diazo-benzidine), Dimethyl Adipimidate, Dimethyl Pimelimidate, Dimethyl Suberaimidate, Disuccininnclyl Suberate, Glutaraldehyde, in-Maleimidobenzoyl-N-Hydroxysuccinimide, Sulfosuccinidyl 4-(N-Maleimidomethyl) Cyclohexane-1-carboxylate etc.

The person skilled in the art is clearly aware that the disposition of the analyte sensor receptor molecule and the analyte sensor molecule may occur at once or successively. Thus, one may first dispose the analyte sensor receptor molecule on the substrate and then add the analyte sensor molecule to the analyte sensor receptor molecule coated supports. Alternatively, one may first establish an interaction between the analyte sensor receptor molecule and the analyte sensor molecule and then dispose this complex on the support. In yet another alternative, one may use a so-called one step reaction in which the analyte sensor receptor molecule and the analyte sensor molecule are brought into contact with the substrate without establishing a complex between the analyte sensor receptor molecule and analyte sensor molecule beforehand.

In a preferred embodiment of the invention, in which an Fc receptor such as ProteinA/G is used as the analyte sensor receptor molecule, an antibody is used as the analyte sensor molecule and a membrane is used as the support, the deposition which may also be designated as coating of the support may be achieved in a one step reaction, i.e. the Fc receptor and the antibody are directly coated on the support being a membrane.

A person skilled in the art is well acquainted with the reaction conditions which have to be regarded when bringing the analyte sensor receptor molecules such as an Fc receptor into contact with a support and when contacting an analyte sensor receptor molecule such as an Fc receptor with an analyte sensor molecule such as an antibody. Typical reaction conditions depend on certain buffers, temperatures, pH conditions etc. However, these conditions will depend on the type of analyte sensor receptor molecule and corresponding analyte sensor molecule as well as on the chemical functionalities that are used in the context of covalent linkage and will usually be known from the literature or provided by e.g. the manufacturer of chemical cross linking agents.

In one embodiment of the present invention, the detection device comprises only one type of analyte sensor receptor molecule and correspondingly one type of analyte sensor molecule being homogeneously distributed on and/or within at least a part of the support.

However, in another embodiment of the present invention which is also preferred, the analyte sensor receptor molecules and correspondingly the analyte sensor molecules may be distributed on and/or within at least a part of the substrate in a spatially ordered and separated manner establishing a support pattern which is usually designated as an “array”.

One of the advantages resulting from disposing the analyte sensor receptor molecules and analyte sensor molecules on a support in an array form is that different types of analyte sensor receptor molecules and correspondingly different analyte sensor molecules can be disposed in a known orientation and distribution on an array. Such an orientation will allow the parallel detection of multiple analytes within a sample.

Typically, such arrays will be made up of spots which represent a specific combination of an analyte sensor receptor molecule and analyte sensor molecule. As the identity of the pairs of analyte sensor receptor molecule and analyte sensor molecule in the spot is known, a complex array can be established. Following industry standards, array formats will have a certain density, meaning that the number of spots ranges from 10 to 100,000, 50 to 50,000, 100 to 10,000 or is 1000, 2000, 3000, 4000 or 5000.

In yet another embodiment, multiple detection devices may be assembled within what is commonly known as a microtitaplate, with each well representing one detection device in accordance with the present invention. Such an assembled detection device may allow performing a number of detection assays in multiples of 96, 384 or 1,536 corresponding to the number of wells of commercially available microtitaplates.

An advantage of these latter aspects of the invention is that miniature support platforms can be developed which permit smaller sample sizes in reaction volumes, which can lead to economies of scale and timesavings. In addition, these analyzers can achieve comparable or greater sensitivity than conventional micro-assay formats.

For disposing the analyte sensor receptor molecules as well as the analyte sensor molecules in a specially arranged array order, one can rely on technologies known from micro array technology such as micro array printing technology etc.

It is to be noted that, in accordance with the present invention, in any of the embodiments described so far, the analyte sensor receptor molecules and analyte sensor molecules are to be present in certain distinct molar ratios and/or number per μm² surface area of the support.

According to the present invention, the molar ratio of the analyte sensor molecules and analyte sensor receptor molecules is between approximately 2:1 and approximately 1:10,000.

In other embodiments of the present invention, which may be preferred, the molar ratio of the analyte sensor molecules and analyte sensor receptor molecules is preferably between approximately 1.9:1 and approximately 1:1000, between approximately 1.8:1 and approximately 1:750, between approximately 1.7:1 and approximately 1:500, between approximately 1.6:1 and approximately 1:250, between approximately 1.5:1 and approximately 1:150, between approximately 1.4:1 and approximately 1:125, more preferably between approximately 1.3:1 and approximately 1:100, between approximately 1.3:1 and approximately 1:90, between approximately 1.2:1 and approximately 1:80, between approximately 1.1:1 and approximately 1:70, between approximately 1:1 and approximately 1:60, even more preferably between approximately 1:1.1 and approximately 1:50, between approximately 1:1.2 and approximately 1:40, between approximately 1:1.3 and approximately 1:30, between approximately 1.4:1 and approximately 1:20 and most preferably between approximately 1:1.5 and approximately 1:15 or between approximately 1:1.5 and approximately 1:10.

Particularly preferred are ratios of 1:1.5 and approximately 1:10.

Equally particularly preferred are ratios of analyte sensor molecules to analyte sensor receptor molecules of 1:1.5, 1:2, 1:3, 1:4, 1:5, 1:6, 1:7, 1:8, 1:9, 1:10, 1:11, 1:12, 1:13, 1:14 or 1:15.

In an exemplary embodiment of the present invention, the number of analyte sensor molecules per μm² surface area of the support may be between 50 and 250,000, between approximately 100 and approximately 100,000, between approximately 150 and approximately 75,000, between approximately 200 and approximately 50,000, between approximately 250 and approximately 25,000, between approximately 300 and approximately 21,000, between approximately 350 and approximately 18,000, preferably between approximately 400 and approximately 15,000, between approximately 450 and approximately 12,000, between approximately 500 and approximately 11,000 and more preferably between approximately 550 and approximately 9000 or between approximately 600 and approximately 8000.

In a particularly preferred embodiment, the number of analyte sensor molecules per μm² surface area of the support may be 600, 700, 900, 1300, 1600, 1900, 2200, 2500, 2800, 3100, 3400, 3700, 4000, 4300, 4600, 4900, 5200, 5500, 5800, 7100, 7400, 7700 or 8000.

In yet another exemplary embodiment of the present invention, the number of analyte sensor receptor molecules per μm² surface area of support may be between approximately 500 and 250,000, between approximately 1000 and approximately 100,000, between approximately 1500 and approximately 50,000, preferably between approximately 5000 and approximately 25,000, between approximately 6000 and approximately 20,000, between approximately 6500 and approximately 16,000, between approximately 7000 and approximately 15,000 and more preferably between approximately 7500 and approximately 13,000 or between approximately 8000 and approximately 12,500.

In a particularly preferred embodiment, the number of analyte sensor receptor molecules per μm² surface area of the support may be 8000, 8500, 9000, 9500, 10,000, 10,500, 11,000, 11,500, 12,000 or 12,500.

Of course, the above mentioned numbers of analyte sensor molecules and analyte sensor receptor molecules per μm² surface area of support may be combined as long as they are within the above specified molar ratios.

The above mentioned molar ratios and molecule numbers per μm² surface area of support particularly apply to a preferred embodiment of the invention in which Fc receptors such as ProteinA, ProteinG and particularly ProteinA/G are used as analyte sensor receptor molecules, antibodies are used as analyte sensor molecules and membranes are used as support. In this context, the molar ratios and number of analyte sensor receptor molecules as well as analyte sensor molecules per μm² of surface area that have been designated above as particularly preferred are to be particularly considered.

In one preferred embodiment, an Fc receptor such as Protein A/G is used as the analyte sensor receptor molecule and an antibody as the analyte sensor molecule with a molar ratio of antibody to Fc receptor between approximately 1:1.5 and approximately 1:10. In this embodiment, the number of antibody molecules may be between approximately 600 and approximately 8000 per μm² surface area of support and the number of Fc receptor molecules may be between approximately 8000 and approximately 125,000 per μm² surface area of support

If e.g. an Fc receptor such as ProteinA/G and an antibody is used, the coating solution of the Fc receptor will typically comprise a concentration of 25-500 nM, 50-250 nM, 75-200 nM or 150 nM, whereas the antibody printing solution will typically be in a concentration of 1 μM, 750 nM, 500 nM, 300 nM, 250 nM, 200 nM, 150 nM, 125 nM, 100 nM, 75 nM, 50 nM, 25 nM, 10 nM, 1.5 nM, 1 nM, 750 fM. A concentration of 100 nM, 150 nM or 200 nM for the Fc receptor and a concentration below 150 nM or below 10 nM for the capture antibody may be preferred in one embodiment. These concentrations are particularly useful if a membrane support of the dimensions of approximately 12.5 cm by 5 cm for the whole membrane (and 1 cm by 1 cm per array) and with porosity of approximately 50 μm to 500 μm, 100 μm to 400 μm, 200 μm or 300 μm is coated.

It is well known that during analyte detection, such as for proteins in a sample, non-specific binding of components of the sample to be tested may occur at the detection device. This non-specific binding may occur to the support, to the analyte sensor receptor molecule and/or the analyte sensor molecule. While one typically will try to take account of such non-specific binding by removing non-specifically bound components after having contacted the detection device with the sample using a so-called washing solution for removing the non-specifically bound components, the inventors of the present invention have found that in one embodiment of the invention it is desirable to treat the detection device with a solution or liquid which is capable of reducing and/or removing and/or preventing non-specific binding to the support, the analyte sensor receptor molecule and/or the analyte sensor molecule. Such solutions for liquids will typically be designated as blocking solutions.

The blocking components which are capable of reducing and/or preventing non-specific binding of sample components to the aforementioned components at the detection device will typically depend on the nature and amount of the support, the analyte sensor receptor molecule and the analyte sensor molecule.

Such blocking components may e.g. comprise detergents such as SDS, TritonX 100, TritonX 80, NP-40, Tween-80, Tween 20 etc. In another embodiment, the blocking components of a blocking solution may be BSA, FSA, HSA, Casein or Fc tails which are not linked to an analyte sensor molecule. Of course, combinations of the above mentioned blocking agents can also be used.

The latter blocking components, namely BSA, HAS, FSA and particularly Casein and Fc tails, are preferred in one of the embodiments of the invention which uses Fc receptors such as ProteinA, ProteinG and particularly ProteinA/G as the analyte sensor receptor molecule, and antibodies as the analyte sensor molecules and membranes as the support.

The blocking solution will typically be applied in the form of a solution or liquid such as a blocking buffer. The further components of such e.g. blocking buffers will be salts, acids etc., depending on the specific use. A typical blocking buffer will be PBS, PH7,4 comprising any of the aforementioned components in a concentration of 1%, 2%, 3%, 4%; 5%, 6%, 7%, 8%, 9%, 10% and up to 15% or 20% by weight of BSA, FSA, HAS or Casein.

Of course, one may also use antibodies as blocking agents as long as the Fc tails thereof are not recognized specifically on the used analyte sensor receptor molecule Fc receptors.

If components such as Casein, BSA, HSA are used as blocking components within a blocking solution, their concentration will typically be between 1 and 15%, 2 and 10% and preferably between 5 and 10%. These concentrations are in percent by weight.

If e.g. Fc fragments or antibodies which are not recognized by the analyte sensor receptor molecules are used as blocking components, the concentration of these latter compounds will typically be between 0.5 to 10 μM, 1 to 5 μM and preferably 1 μM, 2 μM, 3 μM, 4 μM or 5 μM.

In a preferred embodiment, a blocking solution will comprise between 5% to 10% by weight Casein with 1 μM Fc tails as an additive.

The time point of blocking may differ. Thus, a support such as a membrane may be pre-blocked before the analyte sensor receptor molecule such as an Fc receptor is bound to the support. At the same time, blocking may occur if an analyte sensor receptor molecule such as for example ProteinA/G has already been coupled to the membrane before the analyte sensor molecule, in this case being an antibody, is added to the detection device. Blocking may also occur after a complex of analyte sensor receptor molecules such as Fc receptor and an analyte sensor molecule such as an antibody has been formed on a support such as a membrane.

While the above described detection devices have been described largely with respect to Fc receptors as analyte sensor receptor molecules and antibodies as analyte sensor molecules, this should by no means mean that the detection devices are limited to these components. The detection devices in accordance with the invention may be used for a multitude of purposes such as screening small molecule libraries for the presence of a molecule being specific to e.g. a certain protein or enzyme being in this case the analyte sensor molecule. In another embodiment, viruses may be detected in blood samples by their protein or nucleic acid components' capability of binding to specific antibodies which have been immobilized beforehand on the detection devices in accordance with the invention. Similarly, microorganisms in environmental samples or poisonous and environmentally dangerous chemicals therein may be detected using antibodies, receptors or other molecules being capable of specifically interacting with these components.

Another object of the present invention thus relates to a method of detecting at least one analyte in at least one sample, the method comprising the steps of
providing at least one detection device as described above,
contacting said at least one detection device with at least one sample comprising at least one analyte,
optionally washing said at least one detection device with a solution capable of removing non-bound or non-specifically bound samples,
detecting a specific interaction between said at least one analyte sensor molecule and at least one analyte of said at least one sample.

For the purposes of methods in accordance with the invention, detection devices as described may be used. In a preferred embodiment, a detection device is used which uses a membrane as a support, an Fc receptor such as ProteinA, ProteinG and particularly ProteinA/G as the analyte sensor receptor molecule, and an antibody being specific for a certain analyte as the analyte sensor molecule, with the preferred values of molar ratios and number of analyte sensor receptor molecules and analyte sensor molecules per μm² surface area and the blocking components and concentrations being as mentioned above.

These preferred detection devices in accordance with the invention, but also other detection devices, may then be contacted with at least one sample under conditions which allow an interaction between the analyte sensor molecule and the analyte within the sample.

After the sample and the detection device have been brought into contact to allow a specific interaction between the at least one analyte within the sample and the analyte sensor molecule(s) on the detection devices, one may optionally include a so-called washing step which aims at removing and/or reducing non-specifically bound components of the analyte sample which interact non-specifically with the substrate, the analyte sensor receptor molecule and/or the analyte sensor molecule. After this optional washing step, detection of a specific interaction between the analyte sensor molecules and the at least one analyte of the sample will take place.

There are various means of detecting a specific interaction between an analyte within a sample and an analyte sensor molecule. This will be illustrated with respect to an interaction between an antibody as the analyte sensor molecule and a component of a sample such as a protein or a typical chemical compound being specifically recognized by that antibody which may be a component of the sample. However, these explanations are by no means meant to be limited to these exemplary embodiments of the invention.

Detection of the interaction between analyte sensor molecules such as an antibody and the analyte may occur by modifying the analyte with a detectable marker. Modification of the analyte with a detectable marker may take place before contacting the detection device with a sample, during contacting the detection device with the sample or after a specific interaction between the analyte sensor molecule and the analyte has occurred.

Analytes may be modified with detectable markers by modification with e.g. fluorescent components such as Cy5, Cy3, Texas Red, FITC, Attodye, Cydye, Alexa647 etc., radioactive groups etc. If an interaction between the analyte sensor molecule such as an antibody and the e.g. Cy5 labeled analyte occurs, any other labeled components of the sample may be removed by the washing step, and the presence of the specific analyte in the sample may be detected by e.g. a fluorescent signal resulting from the interaction between the analyte sensor molecule and the analyte which is retained on the detection device by way of its interaction with the analyte sensor molecule.

Other methods, such as induced silver staining, may be used for detection. Other detectable markers rely on enzymatic reactions by which staining is produced, for example horseradish peroxidase may be coupled to the analytes in a sample and later on a reaction may be initiated by providing the corresponding substrates leading to staining at the dose positions where an analyte being modified with horseradish peroxidase has interacted with an analyte sensor molecule.

Such enzymatic linkers may further comprise alkaline phosphatase or chemiluminescent systems. Further examples of detectable markers, which are also designated as reporter molecules, include but are not limited to dyes, chemiluminescence compounds, metal complexes, magnetic particles, biotin, hapten, radio frequency transmitters, and radio luminescence compounds.

Other methods of detecting an interaction between the analyte and the analyte sensor molecule may also be used. For example, if the analyte sensor molecule is an antibody, an analyte may be bound specifically to this antibody from the sample. If a washing step is used to remove any unbound or non-specifically bound components from the sample, a further antibody, which is also specific for another portion of the analyte, may be added to the detection device. This antibody may e.g. be linked to a detectable marker. This principle is illustrated in FIG. 1. Such a detectable marker may be a fluorescent marker such as Cy5 as depicted in FIG. 1; however, such a marker may also be e.g. an oligonucleotite of known sequence. If subsequent to the interaction of the so-called secondary antibody with the analyte being retained on the detection device is used for a polymerized chain reaction, the presence of the analyte in the sample may be detected. This so-called immuno PCR is very sensitive and can be used to reliably detect minute amounts of nanolite within the solution. The person skilled in art will know further modifications.

If, as mentioned above, the analyte sensor receptor molecules and analyte sensor molecules are disposed on the support in an array format, the method in accordance with the invention may be used to allow the parallel processing of samples and parallel detection of numerous analytes within one or multiple samples. As each detection spot may correspond to a different analyte sensor receptor and analyte sensor molecule pair being specific for a certain analyte, a signal originating from such a detection spot after such a detection device has been incubated with a sample and processed in the above-described manner will be indicative of the presence of an analyte within a sample.

Thus, one aspect of the present invention relates to the use of detection devices to reliably and specifically detect analytes in a sample while using comparatively low amounts of the rather expensive analyte sensor receptor molecules and analyte sensor molecules. In accordance with the present invention, this can be ascribed to the distinct molecular ratios of analyte sensor molecules to analyte sensor receptor molecules and/or the specific aforementioned number of molecules of analyte sensor molecules and/or analyte sensor receptor molecules per μm² surface area of substrate. Depending on the format of the support, the detection devices in accordance with the invention may be used for the parallel simultaneous detection of multiple analytes within multiple samples.

In the following, the invention is illustrated in view of certain experimental examples. These examples are however in no way meant to limit the scope of the invention. But rather serve to illustrate the invention by way of some of its exemplary embodiments.

### EXPERIMENTS

### Experiment 1

### Determination of Optimal ProteinA Concentration for Coating Membranes

A Biodyne C membrane that comprises COOH groups was used. The membrane had the following dimensions: approximately 1 cm by 1 cm. The membrane was pretreated with 16% EDAC in water for 10 minutes at room temperature. The membrane was then briefly washed with water.

Subsequently, the membrane was coated with FITC-labeled ProteinA in concentrations ranging from 0.001 μM to 10 μM for one hour at room temperature. As a control membrane, a non-coated Biodyne-C membrane was used.

After coating the membrane with the labeled ProteinA, the membrane was blocked with 5% BSA in PBS pH 7.4 for 30 minutes at room temperature. In order to remove unbound ProteinA from the membrane, the membrane was washed 3 times for 5 minutes at room temperature using 1×PBS with 0.05% Tween-20 by volume. Bound labeled ProteinA was measured using a fluorescent microscope with an excitation filter of 480/40 nm and an emission filter of 527/30 nm.

The data shown in FIG. 2 show that the optimal concentration of ProteinA for coating a membrane having dimensions of approximately 1 cm by 1 cm or 12.5 cm by 5 cm was found to have a maximum at 1 μM.

### Experiment 2

### Determination of Antibody Binding Capacity of ProteinA-Coated Membranes Versus Non-Coated Membranes

Two Biodyne-C membranes were used, one of which was coated with ProteinA/G while the other was not coated.

Coating with ProteinA/G was performed by activation of the membrane, as described above, with 16% by weight EDAC for 10 minutes and a subsequent wash in water.

Subsequently, the membrane of the dimensions as described above was coated with 1 μM of ProteinA/G in 1×PBS pH 7.4 for 1 hour at room temperature. The other membrane was mock-treated with a buffer not containing the protein.

The coated membrane was subsequently blocked with 5% BSA in 1×PBS pH 7.4 for 30 minutes at room temperature. This Cy5-labeled Rabbit-anti-Mouse IgG was then printed on the membrane in concentrations ranging from 10 nM to 500 mM. For the second, control membrane, which was not coated with ProteinA/G, the antibodies were used at the same concentrations.

In order to remove any unbound antibodies from the membrane, the membranes were washed for 5 minutes 3 times at room temperature using PBS pH 7,4 with 0.05% by volume Tween-20.

The interaction between the antibody and ProteinA/G was visualized using a fluorescent measurement and quantified.

As can be taken from the data in FIG. 3, the ProteinA/G-coated membrane has a higher capacity for binding antibodies than the non-coated membrane.

### Experiment 3

### Determination of Optimal EDAC Concentration for Linking ProteinA/G to Membrane

The cross-linking agent EDAC is a highly reactive molecule and can react with proteins, being potentially harmful for their conformational functionality. Thus, the purpose was to limit the EDAC concentration as far as possible.

For that purpose, FITC-labeled ProteinA was incubated for 1 hour at room temperature on Biodyne-C membranes and activated with different concentrations of EDAC ranging from 0% to 16% by weight. EDAC was applied in water, under the same conditions as mentioned before. After the incubation, the membranes were blocked with a blocking buffer comprising 5% Casein by weight in 1×PBS pH 7.4 and washed 3 times for 5 minutes with PBS pH 7.4 and 0.05% by volume Tween-20.

From FIG. 4 it can be derived that amounts as low as 1% EDAC already allow for efficient coupling of ProteinA/G to the membrane.

### Experiment 4

### Blocking of ProteinA/G-Coated Membranes

Again, a Biodyne-C membrane was used and coated with ProteinA/G together with a Rabbit-anti-Sheep antibody in a one-step assay as described in examples 1 and 2. As a control, the same type of membrane was only coated with Rabbit-anti-Sheep antibody, but without ProteinA/G.

Subsequently, both membranes were blocked for 1 hour with 5% BSA and 1 μM Biotyn-labeled Rabbit-anti-Mouse antibody in PBS pH 7.4. Subsequently, the membranes were washed 3 times for 5 minutes with PBS pH 7.4 and 0.05% by volume Tween-20.

The ProteinA/G-coated membrane and non-coated membranes were then incubated with Cy5-labeled Streptavidine. If the Biotine-labeled Rabbit-anti-Mouse antibodies had indeed been efficient in blocking ProteinA/G-coated membranes, the ProteinA/G-coated membranes should give stronger signals than the non-coated membranes. This is confirmed in FIG. 5.

Thus, antibodies can be used in a suitable concentration to block ProteinA/G-coated membranes that have been loaded with analyte sensor molecules, such as an antibody.

### Experiment 5

### Further Testing of other Blocking Components

Subsequently, it was tested whether other components may also be suitable for blocking ProteinA/G-coated membranes.

For this purpose, different membranes were incubated with different blocking buffers in different conditions, as can be seen in FIG. 6. In FIG. 6, the different indicated membranes were incubated with a Cy5-labeled Sheep-anti-Mouse antibody. Subsequently, these capture antibody-coated membranes were treated either overnight at 4° C., for 1 hour at room temperature or for 1 hour at 37° C. with blocking solutions consisting of PBS pH 7.4 and the indicated amounts of BSA or Casein.

The results in FIG. 6 clearly show that the optimal blocking compound and optimal blocking concentration is dependent on the specific membrane. However, in general both compounds are found to give suitable results with Casein in this case being somewhat more effective.

### Experiment 6

### Identification of an Optimal ProteinA/G Capture Antibody Ratio

One problem with coating a whole membrane is that a substantial amount of ProteinA/G and correspondingly blocking agent will have to be used. In view of the multivalent and binding mode of ProteinA/G, furthermore binding sites will need to be blocked.

ProteinA/G was incubated before the coating process with the printing solution, e.g. the solution comprising the capture antibody, was carried out. This solution was then used for printing the membrane.

Thus, capture antibodies being Rabbit-anti-Sheep antibodies of a concentration of 10 nM to 800 nM were printed together with 150 nM of ProteinA/G on a Biodyne-C membrane in a printing solution 1×PBS with 5% Glycerol pH 7.4.

The membranes were then blocked for 1 hour at room temperature with 5% Casein, 1 μM of Goat Fc tails in PBS pH 7.4. Subsequently, the capture antibody/ProteinA/G-coated membranes were incubated with the analyte Sheep-anti-Mouse antibody that was labeled with Cy5. This antibody is retained on the membrane by the analyte sensor molecule being a Rabbit-anti-Sheep antibody. After a 1-hour incubation at room temperature, the membrane was washed 3 times for 5 minutes at room temperature with PBS pH 7.4 and 0.05% by volume Tween-20.

The results were then normalized against the Cy5-labeled Sheep-anti-Mouse antibodies that were retained on the coated or non-coated membrane. This normalization was performed as follows: First, the measured intensities are corrected with the background and the excitation intensity. Then the results are normalized against a corrected intensity coming from a Cy5-labeled antibody already present on the membrane. This is a way to correct for the binding capacity of the membrane/ProteinA/G.

The results of the binding efficiency of the analyte, being Cy5-labeled Sheep-anti-Mouse antibody, to the analyte sensor molecule, being Rabbit-anti-Sheep antibody, is depicted in FIG. 7. One can clearly see that at concentrations below 300 nM Rabbit-anti-Sheep antibody, an increased signal is observed for the coated versus the non-coated membrane.

This specific effect opens the possibility of using Fc receptor-coated membranes for retaining capture antibodies on such membranes in a conformational state at distinct ratios and low amount of proteins while retaining a high detection sensitivity.

Thus, if one lowers the number and amount of capture antibodies as well as the concentration of the coating solution for the Fc receptors, an increased number of functional antibodies are observed, resulting in a comparatively stronger signal between the capture antibody and the analyte antibody.

This of course allows miniaturizing detection devices as well as detection methods based on the aforementioned assay principles.

1. A detection device comprising:
at least one support,
at least one analyte sensor receptor molecule which is positioned on and/or within at least a part of said at least one support, and
at least one analyte sensor molecule which is associated with said at least one analyte sensor receptor molecule,
wherein the molar ratio of said at least one analyte sensor and said at least one analyte receptor molecule is between approximately 2:1 and approximately 1:10,000.

2. A detection device according to claim 1,
wherein the molar ratio of said at least one analyte sensor and said at least one analyte receptor molecule is between approximately 1., 9:1 and approximately 1:1000, between approximately 1.8:1 and approximately 1:750, between approximately 1.7:1 and approximately 1:500, between approximately 1.6:1 and approximately 1:250, between approximately 1.5:1 and approximately 1:150, between approximately 1.4:1 and approximately 1:125, between approximately 1.3:1 and approximately 1:100, between approximately 1.3:1 and approximately 1:90, between approximately 1.2:1 and approximately 1:80, between approximately 1.1:1 and approximately 1:70, between approximately 1:1 and approximately 1:60, between approximately 1:1.1 and approximately 1:50, between approximately 1:1.2 and approximately 1:40, between approximately 1:1.3 and approximately 1:30, between approximately 1.4:1 and approximately 1:20, between approximately 1:1.5 and approximately 1:15 or between approximately 1:1.5 and approximately 1:10.

3. A detection device according to claim 2,
wherein the number of said analyte sensor molecules per μm2 surface area of said at least one support is between approximately 50 and 250,000, between approximately 100 and approximately 100,000, between approximately 150 and approximately 75,000, between approximately 200 and approximately 50,000, between approximately 250 and approximately 25,000, between approximately 300 and approximately 21,000, between approximately 350 and approximately 18,000, between approximately 400 and approximately 15,000, between approximately 450 and approximately 12,000, between approximately 500 and approximately 11,000, between approximately 550 and approximately 9000 or between approximately 600 and approximately 8000.

4. A detection device according to claim 2,
wherein the number of said analyte sensor receptor molecules per μm2 surface area of said at least one support is between approximately 500 and 250,000, between approximately 1000 and approximately 100,000, between approximately 1500 and approximately 50,000, between approximately 5000 and approximately 25,000, between approximately 6000 and approximately 20,000, between approximately 6500 and approximately 16,000, between approximately 7000 and approximately 15,000, between approximately 7500 and approximately 13,000 or between approximately 8000 and approximately 12,000.

5. A detection device according to claim 1,
wherein said at least one support is a solid substrate, preferably being selected from the group comprising porous or non-porous materials including polymeric materials, glasses, ceramics, gels, fiber material, non-wovens, metals, filters, membranes and composites thereof.

6. A detection device according to claim 5,
wherein membranes are preferably made of nylon, nitrocellulose, PVDF or Polyethersulfone.

7. A detection device according to claim 1,
wherein said at least one analyte sensor receptor molecule is capable of binding to the Fc portion of an antibody.

8. A detection device according to claim 7,
wherein said analyte sensor receptor molecule is selected from the group comprising ProteinA of Staphylococcus aureus, ProteinG of Streptococci of strains C and G or recombinant ProteinA/G.

9. A detection device according to claim 1,
wherein said at least one analyte sensor molecule is capable of specifically interacting with said at least one analyte sensor receptor molecule by means of one binding site and with an analyte of interest by means of a second binding site, with the binding to the analyte not being substantially influenced by the concomitant binding to the analyte sensor receptor molecule.

10. A detection device according to claim 9,
wherein the analyte sensor molecule is preferably selected from the group comprising proteins, protein receptors, enzymes, antibodies, antigens, aptamers, ligands and haptens.

11. A detection device according to claim 1,
wherein said at least one analyte sensor receptor molecule is disposed on said at least one support by means of a covalent chemical linkage, the substrate providing preferably at least one chemical functional group for coupling of said analyte sensor receptor molecule to said substrate, said at least one chemical functional group being selected from the group comprising carboxyl groups, anhydrides, epoxides, aldehydes, hydrazides, acyl azides, aryl azides, diazo compounds, benzophenone, carbodiimide, imidoesters, isothiocyanates, NHS esters, CNBr, maleimides, tosylates, tresyl chloride, maleic acid anhydrides and carbonyldiimidazole.

12. A detection device according to claim 1,
wherein said detection device is treated with a solution capable of reducing and/or preventing non-specific binding to said at least one support, to said at least one analyte sensor receptor molecule and/or to said at least one analyte sensor molecule.

13. A detection device according to claim 12,
wherein said solution comprises a compound selected from the group comprising BSA, HSA, FSA, Casein, Fc tails or detergents including TritonX 100, TritonX80, Tween 80, Tween 20 and NP-40.

14. Method of detecting at least one analyte in at least one sample, comprising the steps of:
providing at least one detection device according to claim 1,
contacting said at least one detection device with at least one sample comprising at least one analyte,
optionally washing said at least one detection device with a solution capable of removing bound or non-specifically bound samples,
detecting a specific interaction between said at least one analyte sensor molecule and at least one analyte of said at least one sample.

15. Method according to claim 14,
wherein said at least one analyte of said at least one sample is modified with at least one detectable marker.

16. Method according to claim 15,
wherein said at least one detectable marker is selected from the group comprising fluorophores, enzymes, dyes, chemiluminescence compounds, radioisotopes, metal complexes, magnetic particles, biotin, haptens, radio frequency transmitters and radio luminescence compounds.

17. Method according to claim 14,
wherein said method can be used in clinical analysis, identification of novel drugs, blood analysis, drug discovery, structure-functional research, forensics, testing of environmental samples, chemical exposure, testing of small molecule libraries or cell-based assays.
